(19) United States
(12) Patent Application Publication
(10) Pub. No.: US 2011/0213282 A1
(43) Pub. Date: Sep. 1, 2011

(54) Arm sling for post trauma patients

(76) Inventor: Dianne Cortese, Naples, FL (US)

(21) Appl. No.: 12/714,688

(22) Filed: Mar. 1, 2010

(51) Int. Cl. A61F 5/37

(52) U.S. Cl. 602/4

(57) ABSTRACT

An arm sling having broad supporting area on the shoulder of the injured side supports the weight of the upper and lower arm and allows mobility of the upper arm and elbow. The other shoulder from which the non-injured arm depends bears no weight of the supported arm.

### FIELD OF THE INVENTION

The field of the invention is the field of medical support arm slings.

### RELATED PATENTS AND APPLICATIONS

The patents and patent applications referred to herein are hereby incorporated herein by reference in their entirety, including incorporated material.

### OBJECTS OF THE INVENTION

It is an object of the invention to produce an arm sling which is more comfortable to wear than prior art arm slings.

### SUMMARY OF THE INVENTION

The present invention is an arm sling which has a shoulder end and a wrist end. A broad support area of the supporting shoulder from which the supported arm depends bears the weight of the upper and lower arm. Material supporting the arm is prevented from sliding off the shoulder by material passing from the supporting shoulder around the back of the torso of the patient under the other shoulder and connected to the wrist end of the sling. Material supporting the arm is prevented from sliding towards the patients neck by material passing from the shoulder and wrist ends of the sling across the front of the torso, around the front, side, and back of the supporting shoulder, around the back of the torso, underneath the opposite shoulder, to the wrist end of the sling. The other shoulder from which the non-injured arm depends bears no weight of the supported arm.

### BRIEF DESCRIPTION OF THE DRAWINGS

FIG. 1 shows a sketch of the front of a patient with a prior art sling.

FIG. 2 shows a sketch of the back of a patient with a prior art sling of FIG. 1.

FIG. 3 shows a sketch of the front of a patient with the most preferred sling of the invention.

FIG. 4 shows a sketch of the back of a patient with the sling of the invention of FIG. 3.

FIG. 5 shows a sketch of a tube of material for making the sling of the invention of FIG. 3.

FIG. 6 shows a sketch of a folded tube of material of FIG. 3.

FIG. 7. shows a sketch of a tube of material for making the sling of the invention of FIG. 3.

FIG. 8 shows a sketch of a tube of material for making the sling of the invention of FIG. 3.

FIG. 9 shows a sketch of material for making a preferred sling of the invention.

FIG. 10 shows a sketch of material for making a preferred sling of the invention.

FIGS. 11-20 show images of embodiments of the invention.

### DETAILED DESCRIPTION OF THE INVENTION

A sling is usually required for surgical patients who have had operations on their upper bodies such as: rotator cuff repair, total shoulder joint replacement, post humeral fracture, SLAP tear, Biceps repair, open reduction internal fixation (ORIF) of a shoulder, etc.

Patients who may visit a physiotherapist for treatment that would require a sling are usually sent by the therapist to a physician, who may or may not order x rays, prescribe a sling, and prescribe physical therapy treatment. Such cases are usually shoulder dislocation, shoulder fracture, shoulder 2-cuff tear, and shoulder trauma.

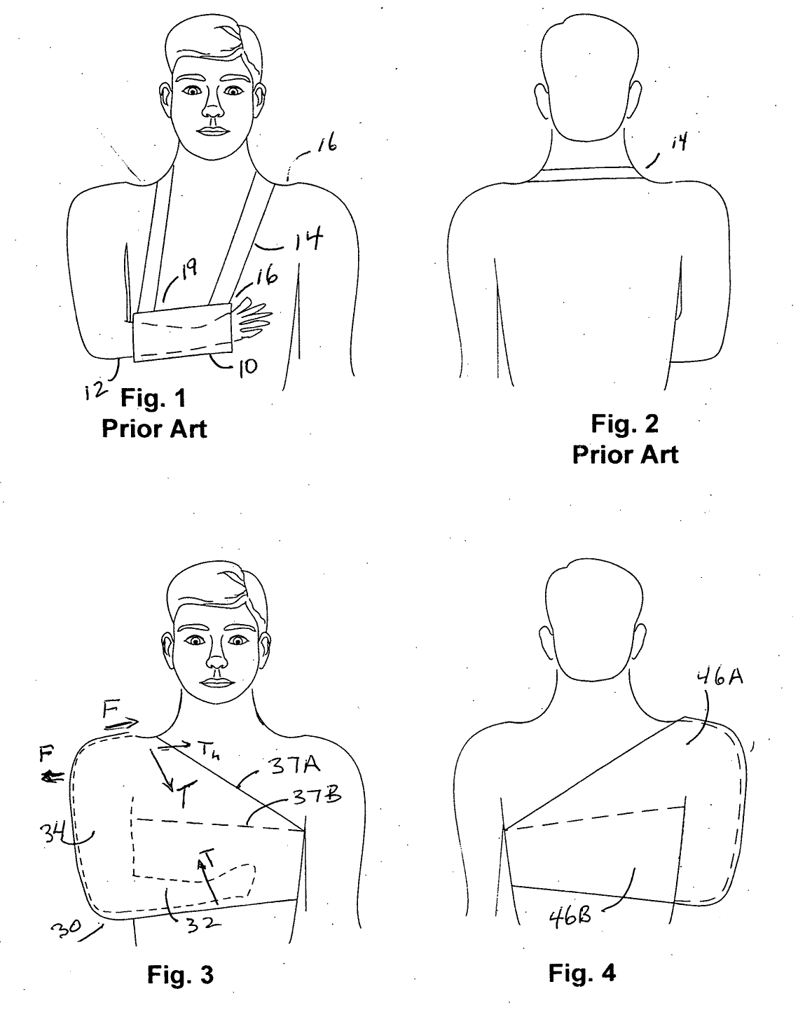

Typical prior art slings such as shown in FIG. 1 and FIG. 2 have a pouch 10 for supporting the injured side forearm 12 and a narrow strap 14 which passes from the wrist end 16 of the pouch 10 over the front of the patient's torso and shoulder 18 on the uninjured side, around the neck of the patient down the front of the torso to the elbow end 19 of a pouch 10. The patient sketched in the front and back views of FIG. 1 and FIG. 2 has the supporting straps in front of the torso on both sides. Another prior art design, not shown, has a closed end of the pouch at the elbow end, and the strap passes from the wrist end at the front of the torso, over the shoulder 18, around the neck and back of the torso underneath the armpit of the elbow from which the supported arm depends to the elbow end of the pouch. The tension force on the strap pulls the strap inwardly towards the neck, and the sling strap may not stay in place and may cause chafing and discomfort on the neck.

Attempts to prevent the sliding and chafing of the sling straps have been made by providing a harness like apparatus having straps over both shoulders which are connected to a belt around the torso of the patient. The straps may cross in the back so that there is some force keeping them from sliding off the shoulder, or the straps may be connected by yet other straps preventing the outward slipping of the straps. These designs are more expensive to produce and to put on and remove, especially when a patient has to do it mostly with one arm and one hand.

One prior art surgical bandage suggested in a US patent filed by Mary New on Jul. 15, 1947 (see FIGS. III and IV of U.S. Pat. No. 2,549,703) is a combination bandage and sling. The bandage part of the sling immobilizes the upper arm against the body. This is disadvantageous for patients who do not require the immobilization of the upper arm and elbow because they are not as free to move the hand on the injured arm to grasp objects or to, for example, type. Such a bandage also precludes using a pillow spacer between the torso and the lower arm and/or elbow which is required after certain operations.

The sling of the present invention shown in FIG. 3 and FIG. 4 comprises a pouch section 30 for supporting the forearm 32 and upper arm 34 of the patient, material denoted 36A and 36B in front of the torso in FIG. 3, and material denoted 46 A and 46B in back of the torso of FIG. 4. Material 36A transmits tension force from the pouch at the elbow, forearm arm, and wrist up to the shoulder on the injured side of the patient. The material 36A loops around under the supported arm of the patient to form a pouch together with material denoted 36B in FIG. 3. The tension force supporting the pouch at the elbow is mostly vertical, and the weight of the upper arm is supported mostly by the front, top, and back of the shoulder. The vertical tension force T in the material 36A supporting the wrist and forearm has a horizontal component Tₕ which keeps the material from sliding outwardly from the neck of the patient and off the shoulder.

The material also passes around outside of the shoulder and the horizontal outward force F of the shoulder acting on the material prevents the material from sliding inwardly towards the neck of the patient. The area of the material exerting force on the shoulder is much higher than the area of the straps and narrower strips of material in the prior art, and the sling therefore exerts much less pressure on the body and is consequently more comfortable.

The tension force in the material must now be supported. In the preferred embodiment shown in FIG. 3, the material pieces 36A and 36B continues from the front of the torso, around the shoulder on the injured side, and passes diagonally around the back of torso of the patient, under the armpit on the uninjured side of the patient, and connects to the pouch at the wrist end of the pouch. In FIG. 4, this material is denoted 46A. Material 36B passes between the torso and the supported arm, around the torso under the armpit of the supported arm, and around the back of the torso to form material 46B, which continues to loop around the torso and connect to the pouch at the wrist end of the pouch. The visible edges of material of the sling of the invention are shown by solid lines, and the edges of hidden material are shown as dashed lines in FIGS. 3 and 4. FIG. 3 shows the material 36A having a visible edge 37A, and the material denoted 36B having a hidden edge 37B. Similarly, material 46A has a visible edge 47A, and material 46B has a hidden edge 47B. Note that the material 37A and 47A forms a band around the torso under both armpits.

The material of the sling is preferably elastic. Spandex® material has been used to good effect.

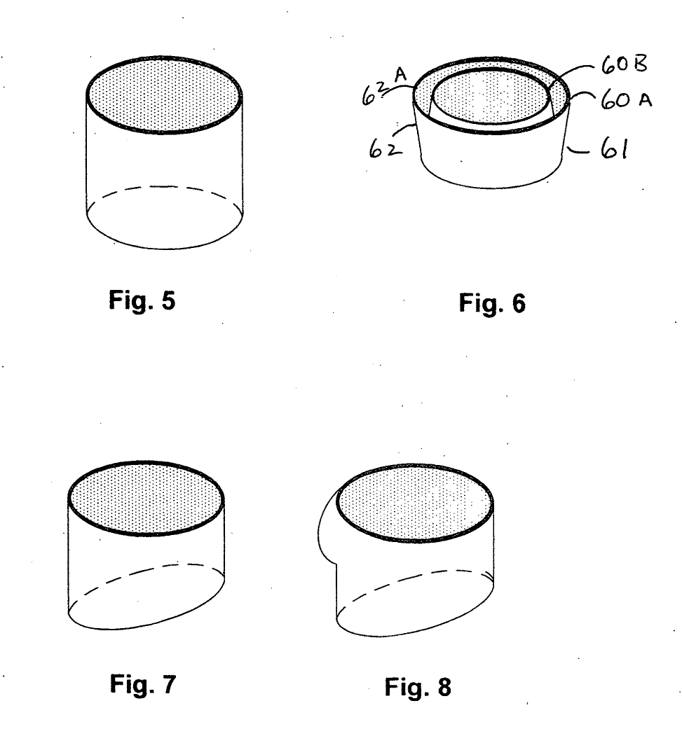

One embodiment of the sling of the invention uses a tube of material sketched in FIG. 5. The tube is conveniently made of spandex material with a single vertical seam (not shown). which will be preferably located under shoulder of the patient on the uninjured side. However, any combination of pieces of material joined together to give a topologically equivalent shape to the tube sketched in FIG. 5 are anticipated by the inventor. In a preferred embodiment, the tube is knitted to give a shape to allow room for the shoulder, arm and elbow of the patient and most preferably knitted with elastic material to allow larger and smaller patients to use slings of just a few sizes. FIG. 6 shows the tube of FIG. 5 folded, with the inner diameter shown reduced from the outer diameter in exaggerated fashion to make the three dimensional sketch of FIG. 6 clearer. In fact, the tube of FIG. 5 could have the top aperture with greater, equal, or less circumference than the bottom. No hidden lines are shown in FIG. 6. The seam is at position 61, The sling is put on by inserting the uninjured arm upwardly through the bottom of the folded tube of FIG. 6 and then putting the bottom of the folded tube over the head. Both the inner and outer parts of the material of FIG. 6 at 60A and 60B are now under the armpit on the uninjured side, and the doubled over material extends downwardly from the armpit. The material edges at 62A and 62B are above the injured shoulder, and resting against the neck. Now, the inner material of the tube of FIG. 6 is pulled downwardly over the injured shoulder until the edge 62 B passes the bottom of the elbow and bottom edge of the supported arm. Then, the edge 62B us raised until the material at 62 B is under the armpit on the injured side. The material edge at 62A remains over the shoulder near the neck on the injured side in the position shown in FIGS. 3 and 4.

The procedure above works for a patient of chest circumference of 110 cm using a uniform cross sectional area tube of spandex of circumference 100 cm and uniform height 60 cm. The spandex is elastic and easily stretches to 120% of its no load dimension. Preferably, a material which has enough elasticity to elastically stretch to 110% of its no load dimension is used. Most preferably, a material which has enough elasticity to elastically stretch to 130% of its no load dimension is used.

Using the uniform tube of FIG. 5 provides excess material under the uninjured side armpit. This material may be removed in more preferred embodiments. More material may also be provided to go over the patients injured side shoulder. FIGS. 7 and 8 show two embodiments incorporating these changes.

The sling of FIGS. 3 and 4 may have one or more apertures (shown later in another embodiment) for allowing the fingers or hand of the patient to protrude. The sling of FIGS. 3 and 4 may have a side aperture (shown later in another embodiment) along the upper arm above the elbow to allow the arm to be withdrawn without removing the sling, and to allow easy insertion of a pillow between the arm and the torso of the patient. The sling of FIGS. 3 and 4 may also have fasteners such as zippers, velcro, buttons, or snaps, for example for fastening a pillow between the sling and the torso of the patient.

The inventor anticipates that elastic material used to produce the sling of the invention may have anisotropic elasticity, as less elasticity is needed in the direction of the axis of the tube of FIG. 5 than around the circumference of the tube. Such a material is sold under the trade name of Spandex® ITY. Preferably, the elasticity of the material used should differ in two dimensions by at least a factor 2. More preferably, the elasticity should differ in two dimensions by at least a factor 4, and most preferably, the elasticity should differ by at least a factor 10.

The embodiments sketched in FIGS. 3 and 4 do not show optional means for increasing or decreasing the length of material encircling the torso of the patient to allow the same sling to fit a the entire range of sizes of possible patients. If the tube of FIG. 6 is cut vertically at location 61 and the material extended in the horizontal direction, the ends may be tied together, or a clasp arrangement may provides a means fitting the tube of FIG. 5 to a patient of larger or smaller torso circumference.

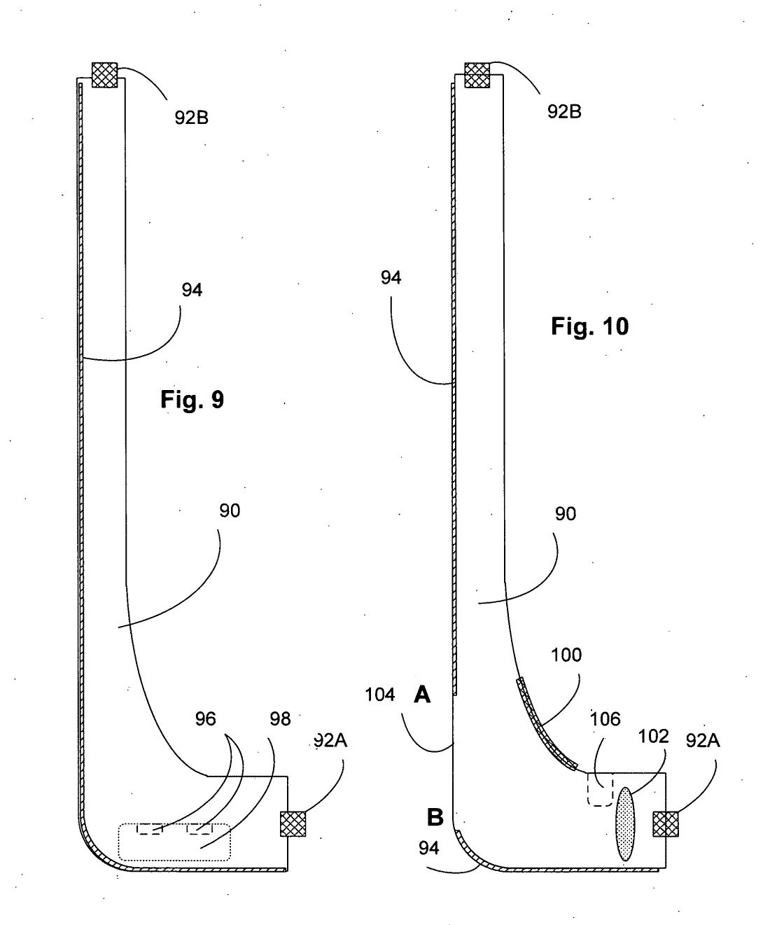

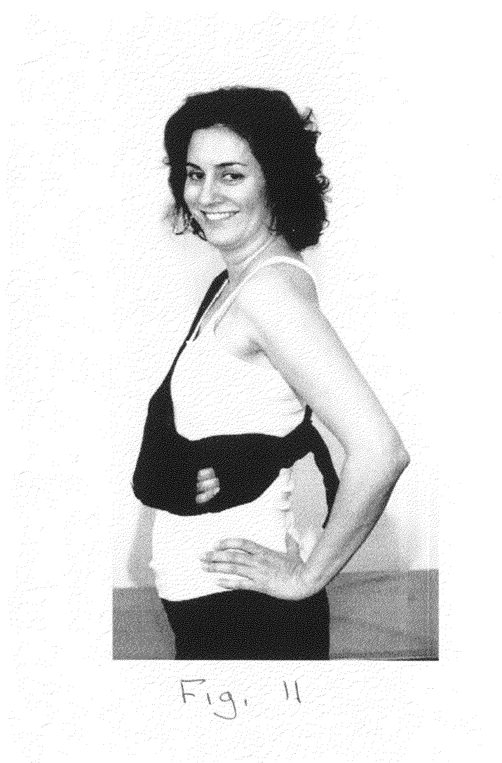

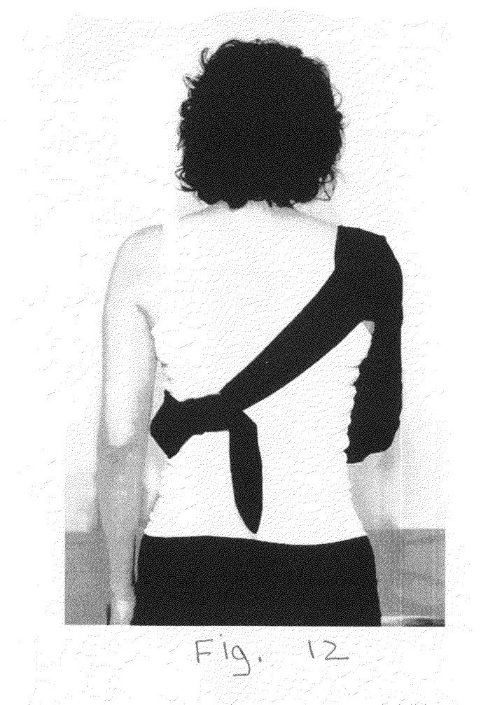

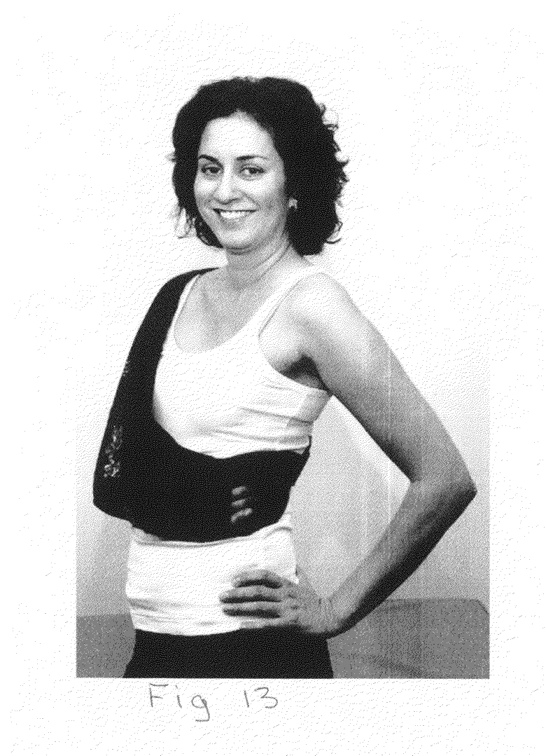

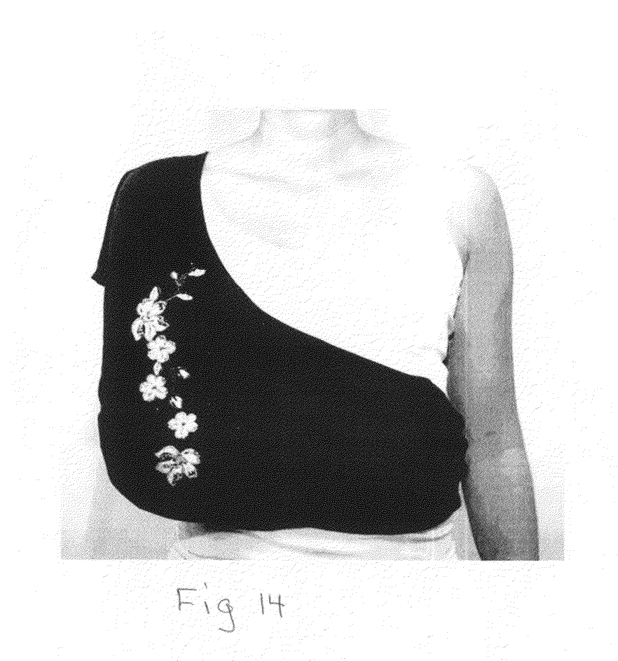

FIGS. 9 and 10 show sketches of material having very little elasticity which may be formed into the sling of the invention, and which are adjustable to fit around the torsos of patients of different chest diameters. FIG. 9 shows a visible top piece 90 and a hidden bottom piece of identical size with mating clasps 92A and 92B. The top piece and bottom piece are joined together by sewing, glueing or other attachment means 94 along one edge. Of course, the sling of the invention may be formed from a single piece of material joined along either the longer straight vertical section of material 90 or the shorter horizontal section of material 90, with the material cut and attached along the remaining section. FIG. 9 shows optional attachment means 96 for attaching a pillow 98 between the arm and the torso. The pillow may be attached inside the pocket of the sling or outside the sling. Attachment means such as a zipper, velcro, snaps, buttons, etc are anticipated by the inventor.

FIG. 10. shows an embodiment where the sling of FIG. 9 has an elastic material 100 attached as shown. The flat material is attached to the stretched elastic material on both the top and bottom pieces of material, and elastic material ensures that the sling is then pulled into position nearer the neck of the patient and ensures that the material 90 covers the front, top, and side of the shoulder. An optional aperture 102 in material 90 for the projection of the hand or fingers of the patient is shown. An optional aperture 104 for entrance and exit of the hand and arm of the patient is shown by the removal of the attachment means 94 from A to B. Also shown on FIG. 10 is a pocket 106 attached on the inside or outside pieces of material to form a pocket sized for carrying small objects like a cell phone, lipstick, keys, etc. Larger objects such as a purse may also be carried in a larger pocket.

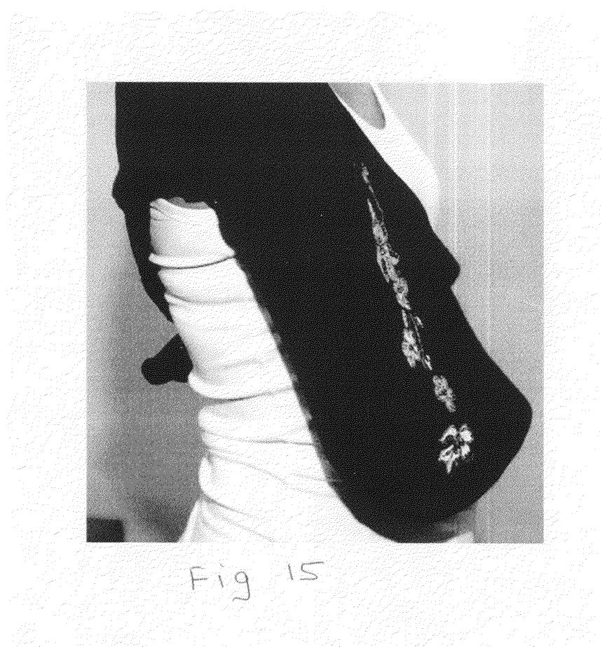

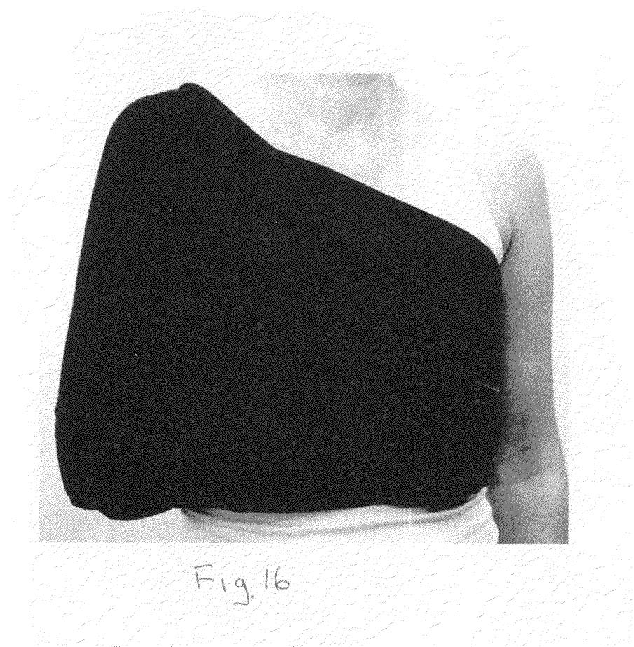

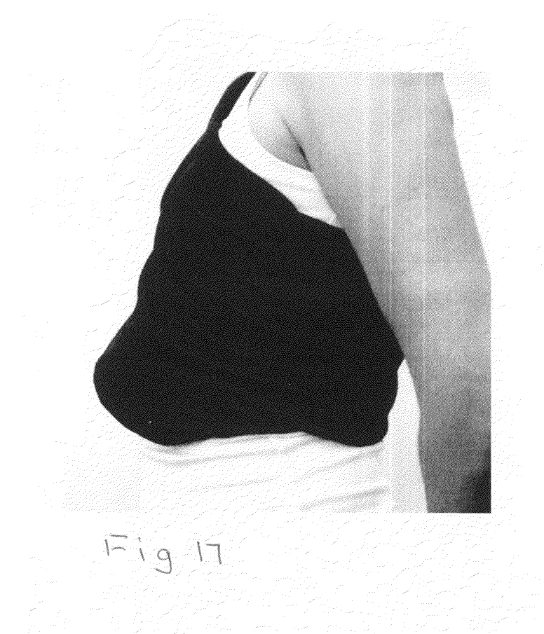

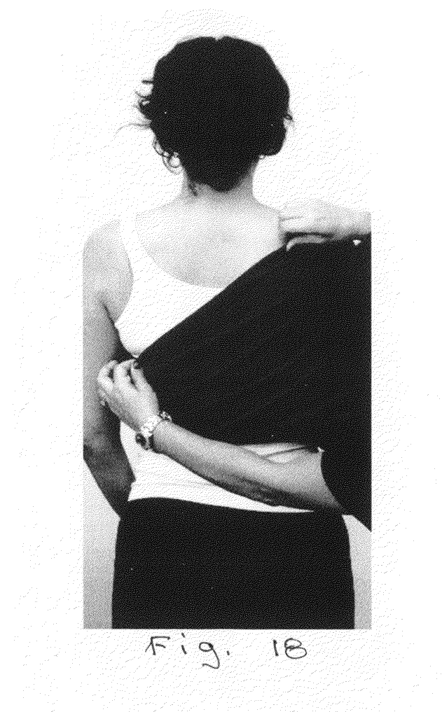

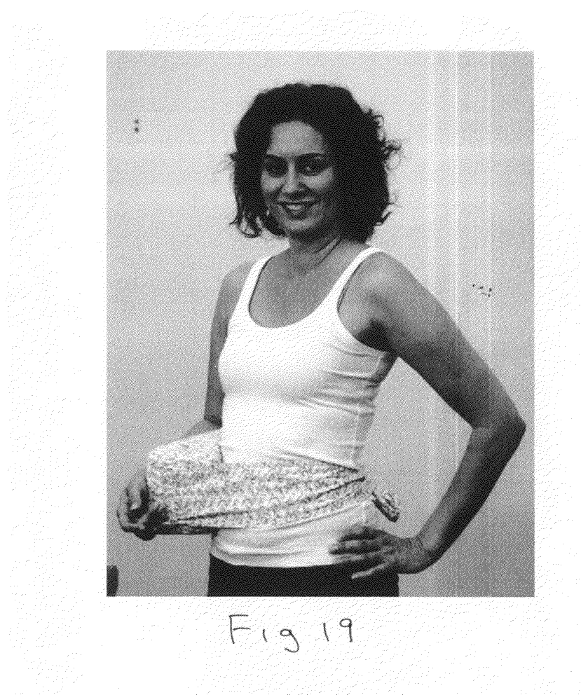

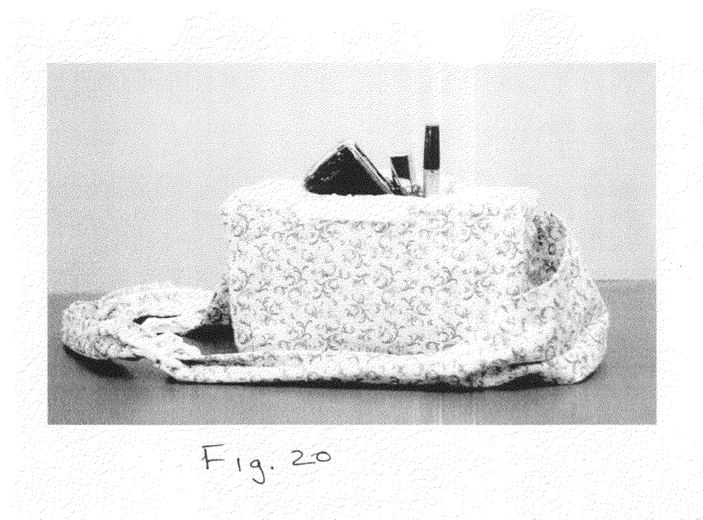

FIGS. 11-20 show images of embodiments of the invention.

Obviously, many modifications and variations of the present invention are possible in light of the above teachings. It is therefore to be understood that, within the scope of the appended claims, the invention may be practiced otherwise than as specifically described.

We claim:

1. A sling for supporting a first forearm and a first upper arm on a first side of a human person, comprising:
a) a pouch section for supporting the first forearm in front of the torso of the human person, the pouch section having a first end for receiving a first elbow connecting the first forearm and the first upper arm, the pouch section having a bottom and a second end for supporting the first forearm and a first wrist attached to the first forearm and a first hand attached to the first wrist;
wherein the human person has a neck, a first and a second shoulder, wherein each shoulder has a top, front, back and outside section away from the neck, and wherein the first upper arm depends from the first shoulder;
b) a support section for supporting the weight of the first forearm and the weight of the first upper arm of the human person, wherein the support section contacts and is supported by the top, front, back, and outside of the first shoulder of the human person, and wherein the weight of the first forearm and the first upper arm of the human person is not supported by the second shoulder of the human person;
wherein the support section is connected by a first connection to the first end, wherein force is transmitted by the first connection from the top, front, and back of the first shoulder to the first end, and wherein the support section is connected by a second connection to the second end, wherein force is transmitted by the second connection from the top, front, back, and outside of the first shoulder around the back of the torso of the human person under the second shoulder of the human person to the second end of the pouch section, wherein the force transmitted by the second connection prevents the support section from sliding outwardly from the neck of the human person and slipping off the first shoulder, and wherein the support section is prevented from sliding inwardly towards the neck of the human person by the contact between the support section and the outside of the first shoulder, and wherein the first upper arm and first elbow are mobile are free to move significantly outwardly and to the back and front with respect to the torso of the human person.

2. The sling of claim 1, wherein the support section, the pouch, and the first and second connections are formed from one continuously knitted fabric material.

3. The sling of claim 1, wherein the support section, the pouch, and the first and second connections are formed from at least one piece of fabric material fastened having edges fastened fixedly together

4. The sling of claim 3, wherein the at least one piece of fabric material is elastic material.

5. The sling of claim 4, wherein the elastic material is an anisotropic elastic material.

6. The sling of claim 1, wherein the second connection is adjustable in length.

7. The sling of claim 1, further comprising exit means for allowing the first hand to protrude from the second end of the pouch.

8. The sling of claim 1, further comprising first attachment means for removably attaching a pillow to the sling in order to place the pillow between the first arm and the torso of the human body.

9. The sling of claim 8, further comprising the pillow, the pillow having second attachment for attaching to the first attachment means.

10. The sling of claim 9, wherein the pillow has a pocket for pocket for carrying personal effects of the human person.

11. The sling of claim 1, further comprising a pocket in the pouch, the pocket for carrying personal effects of the human person.

12. The sling of claim 11, the pocket is correctly sized for carrying a cellular telephone.

13. The sling of claim 11, the pocket is correctly sized for carrying a lipstick.

14. The sling of claim 11, the pocket is correctly sized for carrying a purse.

15. A sling for supporting a first forearm and a first upper arm on a first side of a human person, comprising:
a) a pouch section for supporting the first forearm in front of the torso of the human person, the pouch section having a first end for receiving a first elbow connecting the first forearm and the first upper arm, the pouch section having a bottom and a second end for supporting the first forearm and a first wrist attached to the first forearm and a first hand attached to the first wrist;
wherein the human person has a neck, a first and a second shoulder, wherein each shoulder has a top, front, back and outside section away from the neck, and wherein the first upper arm depends from the first shoulder;
b) a support section for supporting the weight of the first forearm and the weight of the first upper arm of the human person, wherein the support section contacts and is supported by the top, front, back, and outside of the first shoulder of the human person, and wherein the weight of the first forearm and the first upper arm of the human person is not supported by the second shoulder of the human person;
wherein the support section is connected by a first connection to the first end, wherein force is transmitted by the first connection from the top, front, and back of the first shoulder to the first end, and wherein the support section is connected by a second connection to the second end, wherein force is transmitted by the second connection from the top, front, back, and outside of the first shoulder around the back of the torso of the human person under the second shoulder of the human person to the second end of the pouch section, wherein the force transmitted by the second connection prevents the support section from sliding outwardly from the neck of the human person and slipping off the first shoulder, and wherein the support section is prevented from sliding inwardly towards the neck of the human person by the contact between the support section and the outside of the first shoulder, and wherein second connection comprises and elastic material to allow motion of the first upper arm and first elbow outwardly and to the back and front with respect to the torso of the human person.
